(19) United States
(12) Patent Application Publication
(10) Pub. No.: US 2018/0250411 A1
(43) Pub. Date: Sep. 6, 2018

(54) DERMATOLOGICAL COMPOSITIONS AND METHODS USING SAME

(71) Applicant: JACK D. KORBUTOV, PHILADELPHIA, PA (US)

(72) Inventor: JACK D. KORBUTOV, PHILADELPHIA, PA (US)

(21) Appl. No.: 15/912,804

(22) Filed: Mar. 6, 2018

Related U.S. Application Data
(60) Provisional application No. 62/467,641, filed on Mar. 6, 2017.

(51) Int. Cl. A61K 47/34; A61K 47/24; A61K 47/44; A61K 31/573; A61K 9/00; A61P 17/04; A61P 17/06; A61P 17/10

(52) U.S. Cl. CPC A61K 47/34; A61K 47/24; A61K 47/44; A61P 17/10; A61K 9/0014; A61P 17/04; A61P 17/06; A61K 31/573

(57) ABSTRACT

The present invention provides topical compositions that are useful in treating and/or preventing at least one disease selected from the group consisting of atopic dermatitis, eczema, dry skin, baby rash, psoriasis, neuropathic pruritis, acne vulgaris, shingles, hives, sunburn, rosacea and flaky skin in a subject in need thereof.

### CROSS-REFERENCE TO RELATED APPLICATION

The present application claims priority under 35 U.S.C. § 119(e) to U.S. Provisional Application No. 62/467,641, filed Mar. 6, 2017, which application is hereby incorporated by reference in its entirety.

### BACKGROUND OF THE INVENTION

It is well-established in the dermatology field that one of the most effective ways to treat diseases or disorders such as atopic dermatitis and/or pruritis is by occluding the skin with a barrier. Occlusiveness helps treating these diseases or disorders, at least because occluded skin maintains moisture and helps drive any active pharmaceutical ingredient(s) deeper into the dermis (skin layer).

Lotions, creams, and ointments are usually recommended or medically prescribed to offer some occlusion and/or provide active ingredient(s) to the site to be treated. Lotions are the most texturally appealing, because they vanish the fastest. However, they are not as clinically beneficial as creams or ointments because they have little occlusiveness on the skin. Ointments are the most occlusive and clinically desired of the three, but the greasiness and sticky nature of ointments usually reduces the patient's compliance with directions. Creams hit a middle ground of texture and effectiveness, but more severe dermatological disease presentations are difficult to treat without ointments.

As such, there is still a need in the art for shelf-stable topical compositions that provide a desirable texture, and are able to effectively deliver active ingredients to the target site on the skin of the subject. The present invention addresses this need.

### BRIEF SUMMARY OF THE INVENTION

The invention provides a composition. The invention further provides a method of treating a dermatological disease or disorder in a subject using a composition of the invention.

In certain embodiments, the composition comprises at least one active agent. In certain embodiments, the composition comprises at least a cross-linked siloxane rubber. In certain embodiments, the composition comprises at least one silicon-based cyclic compound. In certain embodiments, the composition comprises at least one silicon-based polymer. In certain embodiments, the composition comprises at least one texturizing agent. In certain embodiments, the composition comprises at least one cyclopentasiloxane and dimethicone/vinyl dimethicone crosspolymer.

In certain embodiments, the at least one active agent is at least one selected from the group consisting of mometasone, hydrocortisone, clobetasol, pramoxine, methylcobalamin, cyanocobalamin and/or zinc pyrithione.

In certain embodiments, the at least one cross-linked siloxane rubber is polysilicone-11. In certain embodiments, the at least one silicon-based cyclic compound is cyclopentasiloxane. In certain embodiments, the at least one silicon-based polymer is dimethicone. In certain embodiments, the at least one texturizing agent is hydrophobic. In certain embodiments, the at least one texturizing agent comprises avocado oil.

In certain embodiments, the composition is formulated for topical administration. In certain embodiments, the composition is selected from the group consisting of a cream, an ointment and a lotion.

In certain embodiments, the composition comprises about 50%-90% (w/w) of the at least one cross-linked siloxane rubber. In certain embodiments, the composition comprises about 1%-25% (w/w) of the at least one silicon-based cyclic compound. In certain embodiments, the composition comprises about 0.5%-15% (w/w) of the at least one silicon-based polymer. In certain embodiments, the composition comprises about 0.5%-15% (w/w) of the at least one texturizing agent. In certain embodiments, the composition comprises about 55%-85% (w/w) of the at least one cross-linked siloxane rubber. In certain embodiments, the composition comprises about 2.5%-20% (w/w) of the at least one silicon-based cyclic compound. In certain embodiments, the composition comprises about 1%-10% (w/w) of the at least one silicon-based polymer. In certain embodiments, the composition comprises about 1%-10% (w/w) of the at least one texturizing agent. In certain embodiments, the composition comprises about 60%-80% (w/w) of the at least one cross-linked siloxane rubber. In certain embodiments, the composition comprises about 5%-15% (w/w) of the at least one silicon-based cyclic compound. In certain embodiments, the composition comprises about 2.5%-7.5% (w/w) of the at least one silicon-based polymer. In certain embodiments, the composition comprises about 2.5%-7.5% (w/w) of the at least one texturizing agent. In certain embodiments, the composition comprises about 70% (w/w) of the at least one cross-linked siloxane rubber. In certain embodiments, the composition comprises about 10% (w/w) of the at least one silicon-based cyclic compound. In certain embodiments, the composition comprises about 5% (w/w) of the at least one silicon-based polymer. In certain embodiments, the composition comprises about 5% (w/w) of the at least one texturizing agent. In certain embodiments, the composition comprises about 10% (w/w) of at least one cyclopentasiloxane and dimethicone/vinyl dimethicone crosspolymer.

In certain embodiments, the method comprises administering topically to the skin of the subject a composition of the invention. In certain embodiments, the dermatological disease or disorder is selected from atopic dermatitis, eczema, dry skin, baby rash, psoriasis, neuropathic pruritis, acne vulgaris, shingles, hives, sunburn, rosacea, and flaky skin.

### DETAILED DESCRIPTION OF THE INVENTION

The invention provides a composition that is useful for administering topically at least one active ingredient. In certain embodiments, the composition comprises at least one cross-linked siloxane rubber; at least one silicon-based cyclic compound; at least one silicon-based polymer; and at least one texturizing agent.

The invention further provides methods of treating and/or preventing a dermatological condition comprising contacting and/or administering any one of the compositions of the invention with the skin of a subject in need thereof. In certain embodiments, the dermatological condition contemplated within the invention comprises at least one selected from the group consisting of atopic dermatitis, eczema, dry skin, baby rash, psoriasis, neuropathic pruritis, acne vulgaris, shingles, hives, sunburn, rosacea and flaky skin.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in organic chemistry, formulation chemistry and biology are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, a “disease” is a state of health of a subject wherein the subject cannot maintain homeostasis, and wherein if the disease is not ameliorated then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “effective” means adequate to accomplish a desired, expected, or intended result.

As used herein, the terms “effective amount,” “pharmaceutically effective amount” and “therapeutically effective amount” refer to a nontoxic but sufficient amount of an agent to provide the desired biological result. That result can be reduction and/or alleviation of the frequency and/or severity of signs, symptoms, or causes of a disease, or any other desired alteration of a biological system. An appropriate therapeutic amount in any individual case may be determined by one of ordinary skill in the art using routine experimentation.

As used herein, the terms “inhibiting,” “reducing,” and variations of these terms include any measurable decrease, such as but not limited to complete or substantially complete inhibition.

As used herein, the “instructional material” includes a publication, a recording, a diagram, or any other medium of expression that may be used to communicate the usefulness of the compounds of the invention. In some instances, the instructional material may be part of a kit useful for effecting alleviating or treating the various diseases or disorders recited herein. Optionally, or alternately, the instructional material may describe one or more methods of alleviating the diseases or disorders in a cell or a tissue of a mammal. The instructional material of the kit may, for example, be affixed to a container that contains the compounds of the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

As used herein, the term “or” means “and/or,” unless explicitly indicated to refer to alternatives only or the alternatives are mutually exclusive, although the disclosure supports a definition that refers to only alternatives and “and/or.”

As used herein, the terms “patient” and “subject” refer to a human or a non-human animals. Non-human mammals include, for example, livestock and pets, such as ovine, bovine, porcine, canine, feline and murine mammals. In certain embodiments, the subject is human.

As used herein, the term “personal care composition” refers in a non-limiting manner to products such as cleansers, hair care products (e.g., shampoos, mousses and conditioners) lotions, creams, salves and ointments. These products may be delivered from wipes (e.g., nonwoven substrates), tubes, sachets, patches, pumps or sticks, and the like.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, buccal, mucosal, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 20^{th} Ed., 2000, Easton, Pa.), which is incorporated herein by reference.

As used herein, the language “pharmaceutically acceptable salt” refers to a salt of the administered compounds prepared from pharmaceutically acceptable non-toxic acids including inorganic acids, organic acids, solvates, hydrates, or clathrates thereof. Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, 3-hydroxybutyric, salicylic, galactaric and galacturonic acid. Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “topical” as applied to mode of administration includes but is not limited to “dermal.” The term “dermal” refers to the application of a composition to the skin of a subject. The term “topical” refers to the application of a composition to the body's natural surface, which has not been created by surgical intervention or any artificial means.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

### Compositions

The compositions of the invention are described in a non-limiting manner herein. The compositions of the invention can be in a liquid, solid or viscous form. In certain embodiments, the compositions of the inventions are an ointment, a lotion, a cream, a clear solution, a translucent gel, a soft gel, or any combinations thereof. In other embodiments, the compositions of the invention comprise an emulsion. In yet another embodiment, the compositions of the invention comprise a liposome.

The invention provides a composition comprising at least one active agent; at least one cross-linked siloxane rubber; at least one silicon-based cyclic compound; at least one silicon-based polymer; and at least one texturizing agent.

In certain embodiments, the at least one active agent is at least one selected from the group consisting of mometasone, hydrocortisone, clobetasol, pramoxine, methylcobalamin, cyanocobalamin and/or zinc pyrithione. In other embodiments, the composition comprises about 0.1 mg/g, 0.2 mg/g, 0.3 mg/g, 0.4 mg/g, 0.5 mg/g, 1 mg/mg, 2 mg/g, 4 mg/g, 6 mg/g, 8 mg/g, 10 mg/g, 12 mg/g, 14 mg/g, 16 mg/g, 18 mg/g, 20 mg/g, 22 mg/g, 24 mg/g, 26 mg/g, 28 mg/g, 30 mg/g, 32 mg/g, 34 mg/g, 36 mg/g, 38 mg/g, 40 mg/g, 42 mg/g, 44 mg/g, 46 mg/g, 48 mg/g, 50 mg/g, 52 mg/g, 54 mg/g, 56 mg/g, 58 mg/g, 60 mg/g, 62 mg/g, 64 mg/g, 66 mg/g, 68 mg/g, 70 mg/g, 72 mg/g, 74 mg/g, 76 mg/g, 78 mg/g, 80 mg/g, 82 mg/g, 84 mg/g, 86 mg/g, 88 mg/g, 90 mg/g, 92 mg/g, 94 mg/g, 96 mg/g, 98 mg/g, 100 mg/g, 102 mg/g, 104 mg/g, 106 mg/g, 108 mg/g, 110 mg/g, 112 mg/g, 114 mg/g, 116 mg/g, 118 mg/g, 120 mg/g, 122 mg/g, 124 mg/g, 126 mg/g, 128 mg/g, 130 mg/g, 132 mg/g, 134 mg/g, 136 mg/g, 138 mg/g, 140 mg/g, 142 mg/g, 144 mg/g, 146 mg/g, 148 mg/g, or 150 mg/g of the at least one active agent, or any fraction or multiple thereof. In yet other embodiments, the composition comprises about 0.5-25 mg/g of the at least one active agent. In yet other embodiments, the composition comprises about 1-25 mg/g of the at least one active agent. In yet other embodiments, the composition comprises about 1-50 mg/g of the at least one active agent. In yet other embodiments, the composition comprises about 1-100 mg/g of the at least one active agent. In yet other embodiments, the composition comprises mometasone ranging from, for example, 0.025% to 0.1%. In yet other embodiments, the composition comprises hydrocortisone, for example in the non-limiting range of 1%-2.5%. In yet other embodiments, the composition comprises clobetasol, for example in the non-limiting range of 0.05%-0.1%. In yet other embodiments, the composition comprises pramoxine, for example in the non-limiting range of 1-2%. In yet other embodiments, the composition comprises methylcobalamin, for example in the non-limiting range of 0.05%-0.10%. In yet other embodiments, the composition comprises cyanocobalamin, for example in the non-limiting range of 0.05%-0.10%. In yet other embodiments, the composition comprises zinc pyrithione, for example in the non-limiting range of 0.10%-0.50%.

In certain embodiments, the at least one cross-linked siloxane rubber is polysilicone-11. In other embodiments, the composition comprises about 50%-90% (w/w) of at least one cross-linked siloxane rubber. In yet other embodiments, the composition comprises about 55%-85% (w/w) of at least one cross-linked siloxane rubber. In yet other embodiments, the composition comprises about 60%-80% (w/w) of at least one cross-linked siloxane rubber. In yet other embodiments, the composition comprises about 70% (w/w) of at least one cross-linked siloxane rubber.

In certain embodiments, the at least one silicon-based cyclic compound is cyclopentasiloxane. In other embodiments, the composition comprises about 1%-25% (w/w) of at least one silicon-based cyclic compound. In yet other embodiments, the composition comprises about 2.5%-20% (w/w) of at least one silicon-based cyclic compound. In yet other embodiments, the composition comprises about 5%-15% (w/w) of at least one silicon-based cyclic compound. In yet other embodiments, the composition comprises about 10% (w/w) of at least one silicon-based cyclic compound.

In certain embodiments, the at least one silicon-based polymer is dimethicone. In other embodiments, the composition comprises about 0.5%-15% (w/w) of the at least one silicon-based polymer. In yet other embodiments, the composition comprises about 1%-10% (w/w) of the at least one silicon-based polymer. In yet other embodiments, the composition comprises about 2.5%-7.5% (w/w) of the at least one silicon-based polymer. In yet other embodiments, the composition comprises about 5% (w/w) of the at least one silicon-based polymer.

In certain embodiments, the at least one texturizing agent is hydrophobic. In other embodiments, the at least one texturizing agent comprises avocado oil. In yet other embodiments, the composition comprises about 0.5%-15% (w/w) of the at least one texturizing agent. In yet other embodiments, the composition comprises about 1%-10% (w/w) of the at least one texturizing agent. In yet other embodiments, the composition comprises about 2.5%-7.5% (w/w) of the at least one texturizing agent. In yet other embodiments, the composition comprises about 5% (w/w) of the at least one texturizing agent.

In certain embodiments, the composition further comprises at least one cyclopentasiloxane and dimethicone/vinyl dimethicone crosspolymer. In other embodiments, the composition comprises about 10% (w/w) of the at least one cyclopentasiloxane and dimethicone/vinyl dimethicone crosspolymer.

In certain embodiments, the composition is formulated for topical application. In certain embodiments, the topical composition is selected from the group consisting of a cream, an ointment and a lotion.

Various concentrations of each component contemplated within the invention may be used to generate the composition of the invention. For example, a composition of the invention comprising a component may comprise about 0.1%-99%, 0.1%-60%, 5%-50%, 10%-40%, 5%-25%, 10%-30%, 10%-25%, 25%-50%, 10%-75%, 25%-75%, 10%-65%, 25%-65%, 10%-60%, 25%-60%, 0.1%, 1%, 5%, 10%, 20%, 25%, 30%, 35%, 40%, 45%, 50%, 55%, 60%, 65%, 70%, 75%, 80%, 85%, 90%, 95%, 99% or any range derivable therein, of the component.

In one aspect, the composition of the invention is free of water. In another aspect, the composition of the invention comprises water. In certain embodiments, the composition comprises less than about 5% water. In other embodiments, the composition comprises less than about 2.5% water. In yet another embodiment, the composition comprises less than 1% water. In yet another embodiment, the composition comprises less than about 0.5% water. In yet another embodiment, the composition comprises less than about 0.1% water. In yet another embodiment, the composition comprises less than about 0.05% water. In yet another embodiment, the composition is essentially free of water (substantially anhydrous).

The components of the compositions of the invention may be combined using methods known to those skilled in the art. Non-limiting procedures for preparing the compositions of the invention are provided in the Examples section.

The delivery of the compositions of the invention may be continuous, periodic, a one-time event, or the compositions of the invention may be both periodically administered and continuously administered to the subject on separate occasions.

The invention further contemplates using the compositions of the invention in medical patches. In certain embodiments, the composition is incorporated in a medical patch, which is made of materials that are compatible with the composition. Preparation and use of a medical patch is known to those skilled in the art. The patch comprising the composition may be applied to the skin of the subject, providing localized relief thereof.

In certain embodiments, a preservative or stabilizer is included in the composition. For example, the prevention of the action of microorganisms can be brought about by preservatives such as various antibacterial and antifungal agents, including but not limited to parabens (for example, methylparabens, propylparabens), chlorobutanol, phenol, sorbic acid, EDTA, metabisulfite, benzyl alcohol, thimerosal or combinations thereof. Non-limiting examples of stabilizers which can be included include buffers, amino acids such as glycine and lysine, carbohydrates such as dextrose, mannose, galactose, fructose, lactose, sucrose, maltose, sorbitol, and mannitol. Appropriate stabilizers or preservatives can be selected according to the route of administration desired.

The compositions are in certain embodiments sterile. The composition can be sterilized prior to administration. Methods for sterilization are well known in the art and include heating, boiling, pressurizing, filtering, exposure to a sanitizing chemical (for example, chlorination followed by dechlorination, UV radiation exposure or removal of chlorine from solution), aeration, autoclaving, and the like. A particle filter or microbe filter can be used, and can be necessary according to the route of administration desired

### Methods

The invention includes a method of treating and/or preventing a dermatological disease or disorder in a subject in need thereof. The method comprises administering to the skin of the subject a therapeutically effective amount of a composition of the invention. In certain embodiments, the dermatological disease or disorder is at least one selected from the group consisting of atopic dermatitis, eczema, dry skin, baby rash, psoriasis, neuropathic pruritis, acne vulgaris, shingles, hives, sunburn, rosacea and flaky skin.

In certain embodiments, the composition is applied to the area of the subject's skin that is afflicted with the dermatological disease or disorder. In other embodiments, the area treated with the composition is then covered with a material that prevents significant (or any) loss of the composition by mechanical or chemical removal. In yet other embodiments, the material comprises a cloth, such as but not limited to cotton cloth, such as but not limited to a wet cotton cloth.

The administration of the composition may be continuous, periodic, a one-time event, or the composition may be both periodically administered and continuously administered to the subject on separate occasions.

### Combination Therapies

In one aspect, the compositions of the invention further comprise at least one additional compound that is useful for treating the diseases and/or disorders contemplated within the invention. Non-limiting examples of these compounds include, but are not limited to a steroidal compound and a non-steroidal anti-inflammatory drug (NSAID). In other embodiments, the compound is at least one selected from the group consisting of benzocaine, lidocaine, camphor, capsaicin, menthol, methyl salicylate, pramoxine, and diclofenac. Non-limiting examples of compounds that can be used within the compositions of the inventions are mometasone (ranging from, for example, 0.025% to 0.1%), hydrocortisone (ranging from, for example, 1%-2.5%); clobetasol (ranging from, for example, 0.05%-0.1%); pramoxine (ranging from, for example, 1-2%); methylcobalamin (for example, 0.07%); cyanocobalamin (for example, 0.07%); and/or zinc pyrithione (for example, 0.25%).

A synergistic effect between the at least one active agent and the at least one additional compound can be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Dosing

The amount of the composition of the invention to be administered, for example dermally, depends on the particular indication desired. For example, the dose depends on the type of disease or disorder intended to be treated. The subject's physical characteristics can also be important in determining the appropriate dosage. Characteristics such as weight, age, and the like can be important factors.

The particular dosage may also be dependent on the dosing regime chosen. For example, the composition can be delivered continuously or periodically. Conversely, the composition can be administered as a single administration as a one-time event.

The compositions of the invention can also contain one or more additives, such as a surfactant, PVP, a polymer, an antimicrobial agent, a preservative and so forth. In certain embodiments, it may be desirable to produce a concentrated formulation which can be subject to a final dilution prior to administration.

Local topical administration of composition of the invention may include a carrier vehicle, a topical control release patch, a wound dressing, a hydrocolloid, a foam, or a hydrogel. An appropriate biological carrier or pharmaceutically acceptable excipient may be used.

Those skilled in the art recognize, or are to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

It is contemplated that any embodiment discussed in this specification can be implemented with respect to any method or composition of the invention, and vice versa. Furthermore, compositions of the invention can be used to achieve methods of the invention.

The following examples further illustrate aspects of the present invention. However, they are in no way a limitation of the teachings or disclosure of the present invention as set forth herein.

Other objects, features and advantages of the present invention will become apparent from the detailed description herein. It should be understood, however, that the detailed description and the specific examples, while indicating specific embodiments of the invention, are given by way of illustration only, since various changes and modifications within the spirit and scope of the invention will become apparent to those skilled in the art from this detailed description.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

Described in Table 1 (below) is an exemplary composition of the present invention.

TABLE 1

| Ingredient | Weight (w/w %) |
|---|---|
| Poly silicone-11 | 50-90 |
| Cyclopentasiloxane | 1-25 |
| Dimethicone | 0.5-10 |
| Avocado Oil | 0.5-10 |
| Cyclopentasiloxane and Dimethicone/ | 1-25 |
| Vinyl Dimethicone Crosspolymer | |

Since high occlusion was desired, water was left out of all the iterations evaluated. In certain non-limiting embodiments, water can have a drying effect on skin, and the lack of water slows degradation of active ingredients and also provides the end product with a longer shelf life.

### Example 2

Described in Table 2 (below) is an exemplary carrier of the present invention.

TABLE 2

| Ingredient | Weight (w/w %) |
|---|---|
| Polysilicone-11 | 70 |
| Cyclopentasiloxane | 10 |
| Dimethicone | 5 |
| Avocado Oil | 5 |
| Cyclopentasiloxane and Dimethicone/ | 10 |
| Vinyl Dimethicone Crosspolymer | |

In certain non-limiting embodiments, utilizing the appropriate silicone content is an important element for development of the compositions of the invention.

Different proportions of the silicones were evaluated before arriving at the formula described in Table 2. The carrier described in Table 2 (above) has a soft texture, easy spreadability, and does not lose its place on the skin when exposed to water.

Some of the other iterations were unacceptably thick or runny, and some did not have suitable spreadability. Non-limiting examples are illustrated in Table 3:

TABLE 3

| Ingredient | Weight (w/w %) |
|---|---|
| Polysilicone-11 | 66.67 |
| Cyclopentasiloxane | 12 |
| Dimethicone | 2 |
| Cyclopentasiloxane and Dimethicone/ | 19.33 |
| Vinyl Dimethicone Crosspolymer | |

Comment: thick formulation

| | |
|---|---|
| Polysilicone-11 | 55 |
| Cyclopentasiloxane | 10 |
| Dimethicone | 5 |
| Olive Oil | 20 |
| Cyclopentasiloxane and Dimethicone/ | 10 |
| Vinyl Dimethicone Crosspolymer | |

Comment: greasy formulation

| | |
|---|---|
| Polysilicone-11 | 66.67 |
| Cyclopentasiloxane | 10 |
| Dimethicone | 2 |
| Olive Oil | 11.33 |
| Cyclopentasiloxane and Dimethicone/ | 10 |
| Vinyl Dimethicone Crosspolymer | |

Comment: greasy formulation with unappealing yellow color

| | |
|---|---|
| Polysilicone-11 | 55 |
| Cyclopentasiloxane | 10 |
| Dimethicone | 5 |
| Avocado Oil | 20 |
| Cyclopentasiloxane and Dimethicone/ | 10 |
| Vinyl Dimethicone Crosspolymer | |

Comment: less greasy than prior formulations, but sticky texture on skin

In certain non-limiting embodiments, oil is an important element of the formulations of the invention. Certain oils provide both skin benefits and the ability to carry certain active ingredients. Various iterations were made with different oils at various proportions. Certain oils and certain concentrations resulted in an unacceptably greasy feel or shiny appearance. However, avocado oil at about 5% was determined to provide a suitable texture.

Utilizing the unique combination of silicones and avocado oil in the concentrations described herein provided an elegantly smooth and occlusive base that can be useful for treating atopic dermatitis, pruritis and the like.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A composition comprising at least one active agent, at least a cross-linked siloxane rubber, at least one silicon-based cyclic compound, at least one silicon-based polymer, and at least one texturizing agent.

2. The composition of claim 1, further comprising at least one cyclopentasiloxane and dimethicone/vinyl dimethicone crosspolymer.

3. The composition of claim 1, wherein the at least one active agent is at least one selected from the group consisting of mometasone, hydrocortisone, clobetasol, pramoxine, methylcobalamin, cyanocobalamin and/or zinc pyrithione.

4. The composition of claim 1, wherein the at least one cross-linked siloxane rubber is polysilicone-11.

5. The composition of claim 1, wherein the at least one silicon-based cyclic compound is cyclopentasiloxane.

6. The composition of claim 1, wherein the at least one silicon-based polymer is dimethicone.

7. The composition of claim 1, wherein the at least one texturizing agent is hydrophobic.

8. The composition of claim 7, wherein the at least one texturizing agent comprises avocado oil.

9. The composition of claim 1, which is formulated for topical administration.

10. The composition of claim 1, which is selected from the group consisting of a cream, an ointment and a lotion.

11. The composition of claim 1, which comprises about 50%-90% (w/w) of the at least one cross-linked siloxane rubber; about 1%-25% (w/w) of the at least one silicon-based cyclic compound; about 0.5%-15% (w/w) of the at least one silicon-based polymer; and about 0.5%-15% (w/w) of the at least one texturizing agent.

12. The composition of claim 1, which comprises about 55%-85% (w/w) of the at least one cross-linked siloxane rubber; about 2.5%-20% (w/w) of the at least one silicon-based cyclic compound; about 1%-10% (w/w) of the at least one silicon-based polymer; and about 1%40% (w/w) of the at least one texturizing agent.

13. The composition of claim 1, which comprises about 60%-80% (w/w) of the at least one cross-linked siloxane rubber; about 5%-15% (w/w) of the at least one silicon-based cyclic compound; about 2.5%-7.5% (w/w) of the at least one silicon-based polymer; and about 2.5%-7.5% (w/w) of the at least one texturizing agent.

14. The composition of claim 1, which comprises about 70% (w/w) of the at least one cross-linked siloxane rubber; about 10% (w/w) of the at least one silicon-based cyclic compound; about 5% (w/w) of the at least one silicon-based polymer; and about 5% (w/w) of the at least one texturizing agent.

15. The composition of claim 1, which comprises about 10% (w/w) of at least one cyclopentasiloxane and dimethicone/vinyl dimethicone crosspolymer.

16. A method of treating a dermatological disease or disorder in a subject, the method comprising administering topically to the skin of the subject a composition comprising at least one active agent, at least a cross-linked siloxane rubber, at least one silicon-based cyclic compound, at least one silicon-based polymer, and at least one texturizing agent.

17. The method of claim 16, wherein the dermatological disease or disorder is selected from atopic dermatitis, eczema, dry skin, baby rash, psoriasis, neuropathic pruritis, acne vulgaris, shingles, hives, sunburn, rosacea, and flaky skin.
